(19) United States
(12) Patent Application Publication
(10) Pub. No.: US 2007/0071829 A1
(43) Pub. Date: Mar. 29, 2007

(54) Universally applicable virus inactivated blood plasma produced from portions of non-caucasian plasma

(76) Inventors: Andrea Heger, Wien (AT); Jurgen Romisch, Gramatneusiedl (AT); Tor-Einar Svae, Modling (AT); Wolfgang Marguerre, Heidelberg (DE)

Correspondence Address: JACOBSON HOLMAN PLLC, 400 SEVENTH STREET N.W., SUITE 600, WASHINGTON, DC, 20004, US

(21) Appl. No.: 10/580,548

(22) PCT Filed: Dec. 20, 2004
(86) PCT No.: PCT/EP04/53608

§ 371 Date: Sep. 15, 2006

(30) Foreign Application Priority Data
Dec. 19, 2003 (EP) 03029359.1

(51) Int. Cl. A61K 35/16; C12N 5/08

(52) U.S. Cl. 424/530; 435/372

(57) ABSTRACT

A blood plasma for human use pooled from donors which belong to 10% or more to a non-Caucasian population, the plasma obtainable by mixing blood or blood plasma of blood groups A and B, optionally AB without admixing substantial amounts of blood or blood plasma of blood group 0 characterized in that four to eight parts of blood or blood plasma from donors having the blood group A, more than three parts to seven parts of blood or blood plasma from donors having the blood group B, zero to two parts of blood or blood plasma from donors having the blood group AB.

The present invention relates to a blood plasma pooled from donors which are substantially of non-Caucasians, a pharmaceutical preparation comprising the blood plasma of the invention and the use of the blood plasma of the invention for the manufacturing of a medicament.

### BACKGROUND OF THE INVENTION

Blood groups and the inherent inter-individual differences in human blood were discovered by Karl Landsteiner. The ABO blood group system comprises 4 main phenotypes; O, A, B, and AB, the phenotype being governed by codominant alleles at the ABO locus on chromosome 9.

Transfusion of ABO-identical or compatible plasma, such as FFP of specific blood groups is an effective and generally well tolerated treatment of various types of complex or isolated coagulation factor deficiencies, in thrombotic thrombocytopenic purpura, and In repeated large volume plasma exchange. However plasma transfusion in principle carries some risk of adverse events among recipients, which include both transmission of infectious and non-infectious diseases.

Non-infectious adverse events typically occur when immunologic incompatibility between e.g. transfused donor red blood cells and recipient antibodies produce accelerated destruction of transfused cells. According to Landsteiner's law, any human individual has antibodies in plasma if the corresponding antigen is absent from the red blood cells. For example, by infusing plasma from a group A donor to a group B patient, anti-B antibodies from donors plasma will react with and lead to destruction of the patient's red blood cells. Similarly, plasma from a group B donor, which contains anti-A antibodies, is incompatible with a blood group A patient; and plasma from a group O donor, which contains both anti-A and anti-B antibodies, is incompatible with a patient having blood group A, B, or AB. Therefore, the blood types must be matched to avoid a reaction based on ABO incompatibility.

In addition to non-infectious adverse events, many infectious agents, Including viruses, bacteria, and parasites, can be transmitted through blood transfusion. Well recognized viruses include hepatitis A virus (HAV), hepatitis B Virus (HBV), hepatitis C Virus (HCV), human immunodeficiency virus types 1 and 2 (HIV-1/2), and human parvovirus (PV). The risk of transmission of viral infections is minimized by the introduction of donor screening and new test procedures, and in particular, by the introduction of virus inactivation and/or virus removal procedures. Such procedures include virus inactivation by solvent detergent treatment (EP-A-0 131 740), irradiation, and pasteurization, or virus removal by nanofiltration.

Solvent detergent treated human plasma with specific blood groups, such as Octaplas® of blood groups A, B, O, or AB (Octapharma AG Switzerland), was already developed as an alternative to FFP in order to prevent virus transmission.

Universally applicable plasma in principle can be obtained by using only AB plasma, which contains neither anti-A nor anti-B antibodies (IgM and IgG), thus is compatible with any patient regardless of his blood group. However, the frequency of AB donors (4%) is limited. A plasma suitable for universal transfusion is obtained, if anti-A and/or anti-B antibodies from blood group B and A donors, respectively are removed and/or neutralised by optimal mixing of plasma with the different blood groups. Such neutralization of antibodies was already described (WO-A-99/07390) by mixing 6 to 10 parts of blood or blood plasma of blood group A, 1 to 3 parts of blood or blood plasma of blood group B, and optionally 0 to 1.5 parts of blood or blood plasma of blood group AB without admixing substantial amounts of blood or blood plasma derived from blood group O.

All human races in principle share the same blood system, although the frequency of the four main ABO blood groups varies in populations throughout the world. Measuring the titres of anti-A and anti-B antibodies, it was surprisingly found that not only the frequency of ABO blood groups but also the titers of blood group specific antibodies differ between different ethnic groups. In the Caucasians, in general, the titers of anti-A in group B and group O subjects tend to be higher than the titers of anti-B in group A and group O subjects. On the contrary, in people with non-Caucasian background, such as African-American, Hispanic or Native-American donors, anti-B is almost as high as anti-A titers. Consequently, mixing Caucasian plasma with a considerable portion of non-Caucasian origin at the above mentioned ratios, no optimal neutralization of blood group specific antibodies was found. For example, by mixing of 7 parts of blood group A plasma with 3 parts of blood group B plasma, a considerable portion of which was collected from non-Caucasian donors, high anti-B titres, both of IgM and IgG-type, were found in the plasma pool mixture.

### DESCRIPTION OF THE INVENTION

One object of the invention was to develop a further applicable virus inactivated blood plasma, which is produced by optimal mixing of blood plasma of different blood groups, obtained from blood or plasma of Caucasian origin and portions of non-Caucasian donors, such as donors of African-American, Hispanic and native American origin, facilitating an optimal neutralization of blood group specific antibodies in the mixture.

This object is solved by a blood plasma for human use pooled from donors which belong to 10% or more to a non-Caucasian population, the plasma obtainable by mixing blood or blood plasma of blood groups A and B, optionally AB without admixing substantial amounts of blood or blood plasma of blood group O which comprises
four to eight parts of blood or blood plasma from donors having the blood group A,
more than three to seven parts of blood or blood plasma from donors having the blood group B,
zero to two parts of blood or blood plasma from donors having the blood group AB.

Fractions of blood group O can be present in the plasma of the invention so long as these fractions do not introduce antibodies exceeding substantially the overall A or B blood group antigen concentration.

In the blood plasma product of the invention, ABO blood group specific antibodies are essentially neutralized by free blood group substances by an optimal mix of different blood groups, and therefore, this plasma can be transfused regardless of the patient's ABO blood group. Therefore, the blood plasma of the invention further reduces both, the risk of transfusion related infections as well as ABO incompatibility related fatalities.

In another embodiment of the invention the blood plasma mixture is composed of
five to six parts of blood or blood plasma derived from donors with blood group A,
four to five parts of blood or blood plasma derived from donors with blood group B,
zero to one part of blood or blood plasma derived from donors with blood group AB, and
substantially no blood or blood plasma derived from donors with blood group O.

The ABO blood group specific antibody titre of the blood plasma of the invention is in particular lower than 16 for anti-A and anti-B IgM antibodies, and lower than 64 for anti-A and anti-B IgG antibodies. In another mixture of the blood plasma of the invention, the titre of the anti-A and anti-B IgM antibodies is lower than 8, and the titre of anti-A and anti-B IgG antibodies is lower than 32, employing assays known to a skilled person and described in the European Pharmacopeia (indirect Coombs Test).

Preferably, the blood plasma of the invention is inactivated by the method of EP-A-131740, known as solvent/detergent treatment, irradiation, pasteurisation and/or nanofiltration. A typical solvent/detergent-treatment is for instance use of detergents such as oxyethylated polyphenols, like Triton-X-100, and/or polyoxyethylene derivatives of fatty acids such as Tween 80 and tri-N-butylphosphate (TNBP), or combinations thereof. Also medium to long-chain fatty acids or salts thereof, both saturated and unsaturated, preferably caprylic acid or its salts, can be used for virus inactivation. Other methods are irradiation, pasteurization or nanofiltration. All these methods are known to the person skilled in the art.

Preferably, the blood plasma of the invention is frozen or lyophilized.

The blood plasma of the invention shows coagulation activities comparable to fresh frozen plasma.

The present invention is further illustrated by the following example.

### EXAMPLE 1

190 kg of fresh frozen plasma of blood group A, 156 kg of plasma of blood group B, and 34 kg plasma of blood group AB, all obtained in a considerable portion from non-Caucasian donors, are mixed after thawing at +37° C. The obtained plasma mixture is virus inactivated by using the solvent detergent method. After removal of the virus inactivating reagents and freeze-drying, the amount of free anti-A and anti-B antibodies of both IgM and IgG-type is measured. The titre of anti-A and anti-B antibodies of IgM-type is lower than 8 and the titer of anti-A and anti-B antibodies of IgG-type is lower than 32.

### EXAMPLE 2

205 kg of fresh frozen plasma of blood group A, and 137 kg of plasma of blood group B, all obtained in a considerable portion from non-Caucasian donors, are mixed after thawing at +37° C. The same procedure as in example 1 was used. The titre of anti-A and anti-B antibodies of IgM-type are lower than 8 and of IgG-type lower than 32.

1. A blood plasma for human use pooled from donors which belong to 10% or more to a non-Caucasian population, the plasma obtainable by mixing blood or blood plasma of blood groups A and B, optionally AB without admixing blood or blood plasma of blood group 0 characterized in that
five to six parts of blood or blood plasma from donors having the blood group A,
four parts to five parts of blood or blood plasma from donors having the blood group B,
zero to one part of blood or blood plasma from donors having the blood group AB.

2. The blood plasma according to claim 1 virus-inactivated by any virus inactivation or virus removal method.

3. The blood plasma according to claim 2 wherein the blood plasma was inactivated by solvent/detergent treatment, irradiation, pasteurisation and/or nanofiltration.

4. The blood plasma according to claim 3 wherein the virus inactivation was performed by using detergents such as oxyethylated polyphenols, like Triton-X-100, and/or polyoxyethylene derivatives of fatty acids such as Tween 80 and tri-N-butylphosphate (TNBP), or combinations thereof.

5. The blood plasma according to claim 3 virus inactivated by treatment with long-chain fatty acids, such as caprylic acid or the respective salts.

6. The blood plasma according to claim 1 substantially free of virus inactivating agents.

7. The blood plasma of claim 1 having ABO blood group specific antibody titre lower than 16 for anti-A and anti-B IgM antibodies, and lower than 64 for anti-A and anti-B IgG antibodies.

8. The blood plasma of claim 1 in liquid, frozen, dried, or lyophilised form.

9. A pharmaceutical composition comprising the blood plasma of claim 1.

10. Use of the blood plasma of claim 1 for the manufacturing of a medicament for the treatment of coagulation factor deficiencies, thrombotic purpura, and in repeated large volume plasma exchange.

11. A process for manufacturing the blood plasma of claim 1 by admixing
four to eight parts of blood or blood plasma from donors having the blood group A,
more than three parts to seven parts of blood or blood plasma from donors having the blood group B,
zero to two parts of blood or blood plasma from donors having the blood group AB.
